(19) United States
(12) Patent Application Publication
(10) Pub. No.: US 2024/0136069 A1
(43) Pub. Date: Apr. 25, 2024

(54) Medical Voice Bot

(71) Applicant: BIOTRONIK SE & Co. KG, Berlin (DE)

(72) Inventor: Matthias WENZEL, Bremen (DE)

(73) Assignee: BIOTRONIK SE & Co. KG, Berlin (DE)

(21) Appl. No.: 18/546,523

(22) PCT Filed: Feb. 22, 2022
(86) PCT No.: PCT/EP2022/054375

§ 371 Date: Aug. 14, 2023

(30) Foreign Application Priority Data
Mar. 12, 2021 (EP) 21162224.6

(51) Int. Cl. G16H 50/30; A61B 5/00; A61B 5/08

(52) U.S. Cl. CPC G16H 50/30; A61B 5/08; A61B 5/4803

(57) ABSTRACT

The present invention relates to determining a deterioration of a health state of a patient. A system comprises: a) means for receiving a first acoustic input, associated with the health state of the patient at a first time, and a second acoustic input, associated with the health state of the patient at a second time; b) means for determining whether the health state of the patient has deteriorated based at least in part on a comparison of the first acoustic input with the second acoustic input; c) means for indicating the deterioration of the health state of the patient, if it is determined that the health state of the patient has deteriorated.

### CROSS-REFERENCE TO RELATED APPLICATIONS

This application is the United States National Phase under 35 U.S.C. § 371 of PCT International Patent Application No. PCT/EP2022/054375, filed on Feb. 22, 2022, which claims the benefit of European Patent Application No. 21162224.6, filed on Mar. 12, 2021, the disclosures of which are hereby incorporated by reference herein in their entireties.

### TECHNICAL FIELD

The present disclosure relates to systems and methods for determining a deterioration of a health state of a patient, such as implemented by an artificial intelligence (AI) based system, e.g., a medical voice bot.

### BACKGROUND

It is one of the main goals of modern medicine to maintain the health state of a patient or to recover the health state of a patient after suffering from a disease. Another very important aspect of modern medicine may be seen in early diagnostics of diseases, e.g., the early determination of a potential deterioration of the health state of a patient.

It is a typical problem that a deterioration of the health state of a patient is not discovered early enough to be able to reverse the deterioration of the health state, ideally on a short time scale, and to recover a satisfying health state of the patient. In particular, many diseases are (only) curable if discovered in an early stage, particularly some health state deteriorations are likely to be fatal, unless discovered early. However, sometimes the corresponding indicators are unclear and/or not specific and may thus easily be overlooked and/or ignored with potentially severe implications on the further evolution of the health state of the patient.

Therefore, there is a need to improve the known ways to determine a deterioration of the health state of a patient early on.

The present disclosure is directed toward overcoming one or more of the above-mentioned problems, though not necessarily limited to embodiments that do.

### SUMMARY

According to an aspect of the present disclosure, the above need is at least partly met by a system for determining a deterioration of a health state of a patient. The system may comprise means for receiving a first acoustic input, associated with the health state of the patient at a first time, and a second acoustic input, associated with the health state of the patient at a second time. It may further comprise means for determining whether the health state of the patient has deteriorated based at least in part on a comparison of the first is acoustic input with the second acoustic input. Additionally, the system may comprise means for indicating the deterioration of the health state of the patient, if it is determined that the health state of the patient has deteriorated.

In some examples, the means for determining may additionally or alternatively be adapted to determine whether the health state of the patient has deteriorated based at least in part on a comparison of the first acoustic input with at least one other acoustic input.

The system according to the present invention may hence in particular provide a continuous (re-)evaluation of the current health state of the patient based on current acoustic inputs (i.e., based on current data). By receiving acoustic inputs that originate from subsequent instants in time, it may be possible to determine a deterioration of the health state of the patient earlier on, e.g., according to a predetermined period or frequency, or even in real-time, based on patient-specific changes between the first acoustic input and the second acoustic input which relate to different instances in time. In particular, the means for determining may use artificial intelligence (AI). Also, the system may be implemented without dedicated (additional) high-cost measurement devices, but may be implemented using a smartphone (or any other user device such as e.g., a smart home device (e.g., a smart speaker, a smart TV, etc.)), harnessing easily obtainable acoustic input which may, however, be a decisive factor in assessing the health state.

Since the system may entirely operate without external input of a medical provider (e.g., a doctor, a nurse, etc.), the overall operational costs may also be minimized, and at the same time enable a 24/7 health surveillance of the patient. This may facilitate that certain patients, which require such care-taking, may live at home instead of living in hospitals or rehabilitation facilities. Hence, patients may be discharged from hospitals early, even though they still require regular check-ups or even permanent surveillance. Patients may return home, and at the same time an adequate surveillance of the health state of the patient may be provided such that a possible deterioration of the health state may be discovered in an early stage.

Also, the acoustic inputs such as e.g., a spoken sequence may be used with minor efforts for the patient such that additional onus on patients is minimized. No additional technical devices which may require sophisticated settings may be needed. This may particularly be beneficial for older patients that would require regular medical check-ups for preemptive care taking that may be easily forgotten (or the patients may be unable to reach a doctor due to already pre-existing conditions). What is more, the determination based on the acoustic input may be enhanced by using additional vital parameters associated with the health state (e.g., as further be elucidated below) which may be obtained from e.g., a smartphone, smartwatch or similar devices.

The system according to the present disclosure may comprise a microphone, a speaker, an AI unit and one or more interfaces as described herein, e.g., to a network.

The system according to the present disclosure may be comprised by a user device (e.g., a mobile device, such as e.g., a smartphone, a wearable, e.g., a smartwatch, a medical device, e.g., a stationary or dedicated wearable device, an implant, etc.). The system may be implemented as such a device or may be part of such a device (e.g., the system may be implemented as an app and/or an integrated circuit). Such a device may comprise a microphone, processing electronics (e.g., at least one of a processor, a transient and/or non-transient memory, a bus system, means for indicating a potential deterioration of the health state of the patient), speakers and generically means for indicating the deterioration (such as a speaker, and/or a screen, and/or an optical or acoustic alarm).

The system may also be implemented as a server-based system. The system may be understood as at least one of a Software as a Service (SaaS), a Hardware as a Service (HaaS) and/or a Infrastructure as a Service (IaaS) solution. The server-based system may implement the system such that it is or is part of a cloud-based system.

For example, if the system is implemented such as to be comprised by a user device, the means for receiving the first and/or the second acoustic input may comprise a microphone, wherein the microphone may be used to record the first and/or the second acoustic input. The microphone may be a stand-alone microphone (e.g., in connection with the user device) and/or may be part of the user device (e.g., part of a smartphone, a wearable (such as e.g., a smartwatch) and/or a smart home device (such as e.g., a TV, a smart speaker and/or any other device which comprises a microphone)). If the system is exemplarily implemented as an app, the app may receive the first acoustic input and/or the second acoustic input by means of an implemented software interface (e.g., a web socket and/or any other suitable software interface). The microphone may also be part of a medical device which may either be a stationary and/or a mobile device (such as a hearing aid device or an implant etc.).

Moreover, it may also be foreseen that the user device does not record the first acoustic input and/or the second acoustic input itself, e.g., it does not necessarily need to comprise a microphone. The user device may receive the first acoustic input and/or the second acoustic input from other sources. For example, the means for receiving, of the user device, may comprise an interface. The interface may facilitate a wired (e.g., ethernet, USB, etc.) and/or a wireless (e.g., Wi-Fi, LTE, 3G, 4G, 5G, Bluetooth, RFID, NFC, etc.) connection. The first acoustic input and/or the second acoustic input may be received from a remote storage device (e.g., a database, a blockchain, a cloud) and/or at least one other (user) device (e.g., another smartphone, a wearable, etc., etc.). The interface may allow the reception of the first acoustic input and/or the second acoustic input, e.g., by means of a stream (i.e., recoding of the first acoustic input and/or the second acoustic input occur in real time), e.g., from another user device or any other suitable device which is capable of providing the first acoustic input and/or the second acoustic input, and/or as a pre-recorded data package (i.e., the first acoustic input and/or the second acoustic input is not received in real time but the first input and/or the second acoustic input has been recorded first and is then received with a delay).

In case the system is implemented as a server-based system the means for receiving may be implemented as an interface (as outlined above). By means of the interface, the system to may receive the first acoustic input and/or the second acoustic input from any other device e.g., a user device or any other suitable device which is capable of providing the first acoustic input and/or the second acoustic input. The interface may provide a connection to e.g., a user device which comprises a microphone or which already stores the first acoustic input and/or the second acoustic input, and from which the system may receive the first acoustic input and/or the second acoustic input. Additionally or alternatively, the system may also be connected to a stand-alone microphone by means of the interface and may therefore receive the first acoustic input and/or the second acoustic input from the microphone directly. In addition or as an alternative, it may also be possible that the system is connected to a database (e.g., locally or remotely), similarly as outlined above.

In any implementation, it may also be foreseen that the patient is provided with means for enabling or disabling the means for receiving, e.g., for confirming an activation of the microphone (e.g., by means of pressing a hardware and/or software button or by a vocal confirmation). This may provide an increase in privacy experience for the patient.

Independently of the implementation of the system, it may also be possible that more than one microphone is used (as part of the system (e.g., in a user device) and/or in connection with the system (user device or server-based)) to record the first acoustic input and/or the second acoustic input. In such a case a plurality of microphones may be used (which may at least in part be part of the system, e.g., a smartphone) and additional noise cancellation effects may be provided. Such noise, which may disturb the determination whether the health state of the patient has deteriorated, may e.g., originate from construction work, fans, wind, children, traffic, etc. By measuring the voice of the patient at various locations in a room, such external contributions may be filtered prior to generating the first acoustic input and/or the second acoustic input, facilitating a reliable determining of the health state.

All interfaces may facilitate a secure connection, e.g., by encrypting the transmitted data (i.e., the first and/or the second acoustic input and/or any other transmitted data). This ensures the privacy of the patient.

The apparatus may comprise means for providing the patient with at least one question. In response to the question, the patient may answer, wherein the answer may be understood as the first acoustic input and/or the second acoustic input and/or an additional acoustic input. The at least one question may be provided to the patient once or may be provided to the patient on a periodic basis (e.g., every minute, every hour, every day, every week, every month, every year, etc.). The question may relate to the health state of the patient (e.g., the subjective feeling, pain, tachycardia, current fitness level, medication, liquid consumption, alcohol consumption, diet, etc.). Additionally or alternatively, the questions may also relate to non-medical topics such as questions concerning the weather. The means may comprise e.g., a graphical user interface, e.g., a touchscreen, and/or an acoustic interface, e.g., a loudspeaker in case of a user device implementation. Additionally or alternatively, the means may comprise an interface that is also used to receive acoustic input data from the patient (e.g., a user device).

The means for providing the question may comprise an AI. The AI may e.g., select and/or generate questions, e.g., based on an analysis of the first and/or second acoustic input and/or based on historic health states. In response to the question, e.g., a push notification on the display of a smartphone and/or an articulation of the question by means of a smart speaker, a smartphone and/or any other suitable device may activate a respective microphone to record the respective acoustic input, which may be the first acoustic input and/or second acoustic input.

The acoustic input, associated with the health state of the patient, may relate to a spoken sequence of the patient and/or any respiratory sound (as it will further be described below) which may e.g., be a breathing sound of the patient, a cough sound of the patient and/or any other sound of the patient which may be related to the health state of the patient. It may be possible to store the acoustic raw data or only the relevant parameters which are derivable from the acoustic raw data (as it will be explained below). The corresponding data may be stored locally on the system and/or remotely (e.g., in a database, blockchain, etc.).

The first and the second acoustic input of the patient may relate to two acoustic inputs which were recorded subsequently. Subsequently may be understood as referring to two recordings at a certain time interval apart from each other. Such a time interval may refer to several seconds, several minutes, several hours, several days, several weeks, etc. In other words, the first and the second acoustic input may have been recorded at two different instances in time. The first and the second acoustic input may simultaneously be received at the system or the second/first acoustic input may be received after a certain time interval has elapsed after the reception of the first/second acoustic input.

In some situational contexts it may also be beneficial that the first acoustic input and/or the second acoustic input are recorded more than once (e.g., twice) at each time but by different microphones (and, optionally, different devices). In such a case, a redundancy of the recording may be ensured. In case a poor connection between device and the at least one microphone occurs, this may prevent a full data loss associated with both the first acoustic input and the second acoustic input. Moreover, if one of the at least one microphone is located in a noisy environment (such as e.g., close to an open window), the recording by said microphone may be of poor quality due to environmental noise (e.g., a busy street, construction work, etc.) and thus not adequate for the determination of the deterioration of the health state. If so, the recording of that microphone may be rejected, but the remaining recording may still be used for determining whether the health state of the patient has deteriorated. As outlined above, also noise cancellation may be used when the first and/or second acoustic inputs are each based on recordings by several microphones.

The first and/or the second acoustic input may additionally be provided with a time stamp, which may e.g., be stored as meta data in the data package associated with the first acoustic input and/or the second acoustic input. The time stamp may relate to the time and/or date at which the first acoustic input and/or the second acoustic input was recorded and/or at which the first acoustic input and/or the second acoustic input was received at the system according to the present disclosure. This may in particular facilitate a later reconstruction of a potential deterioration of the health state of the patient vs. time.

It may also be possible that more than two acoustic inputs are received, i.e., a third acoustic input, a fourth acoustic input, etc. In such a case, a prolonged tracking of the health state of the patient may be enabled, e.g., once a week over a time span of e.g., one year. A tracking of the health state of the patient may refer to repeatedly receiving acoustic inputs. Repeatedly receiving acoustic inputs may refer to periodically receiving acoustic inputs, e.g., every second, every minute, every hour, every day, every month, every year, etc. Alternatively and/or additionally, the acoustic inputs may be received non-periodically, e.g., based on an external request (e.g., by means of a respective request from a health care provider which may be initiated remotely) and/or by means of an actuation of a button/switch by a patient (e.g., in response to a subjective feeling that the health state of the patient may have deteriorated) or based on other parameters as outlined further below. For example, if the system is implemented in an app, it may provide means for setting whether acoustic input is to be provided continuously or periodically, and in the latter case it may allow setting a frequency.

The association of an acoustic input with the health state of the patient may relate to an encoding of the health state of the patient by a certain parameter in the acoustic input. In other words, at least one parameter may be determined from, e.g., the first acoustic input and/or the second acoustic input which may be suitable for establishing a conclusion concerning the current health state of the patient. For example, a pitch of the voice or an unusual noise in a respiratory sound, and/or any other acoustic parameter may be used. For example, if the patient suffers from cardiac insufficiency, the patient is at risk to additionally suffer from wateriness in different tissues (e.g., the lung). As a consequence, said wateriness may result in a characteristic breathing sound which may be determined in the first and/or the second acoustic input. The accumulation of liquid, e.g., in the lung of a patient with a precondition of cardiac insufficiency may hence be detected early on.

In some examples, the system may be dedicated for monitoring patients with cardiac insufficiency and/or chronic respiratory diseases.

The means for determining a deterioration of the health state of the patient may refer to a comparison of the first acoustic input with the second acoustic input. Such a comparison to may relate to the comparison of at least one parameter which may be derived from each of the first acoustic input and the second acoustic input. Such a parameter may e.g., be a pitch of a voice of the patient as it will further be described below. For example, the first acoustic input may have been recorded upon or shortly after a discharge of the patient from a hospital when the health state of the patient was considered as satisfactory (e.g., based on is a subjective feeling of the patient and/or a judgement of the health care provider). A health care provider may be understood as a member of a medical staff (e.g., a doctor, a nurse, etc.) and/or any rehabilitation related member and/or a home nurse. After e.g., one day, one week or one month after discharge from the hospital, a second acoustic input may be recorded. Both the first acoustic input and the second acoustic input may be supplied to the system according to the present invention. The respective pitches of the voice of the patient (as represented by the first acoustic input and/or the second acoustic input) may be calculated (as it will be described in further detail below) and compared to each other. If a difference between the first and the second acoustic input is determined, the difference may be interpreted as a change of the health state of the patient. A deterioration of the health state of the patient may be based on the definition of a threshold. For example, if the difference of the at least one parameter (as derived from the first acoustic input and the second acoustic input) is larger than a certain threshold, the difference may be interpreted as a deterioration of the health state of the patient. The threshold, which may be used to define a deterioration of the health state of the patient may e.g., be defined by a health care provider, the patient, etc.

It may also be possible, that the first acoustic input and/or the second input are compared to at least one other acoustic input. The at least one other acoustic input may relate to at least one stored acoustic input which may be at least one historic acoustic input. The at least one stored acoustic input may have been recorded in the past (e.g., at least a day ago, at least a week ago, at least a month ago, at least a year ago) and may exemplarily relate to a historic health state (which may additionally be defined as healthy or abnormal). For example, if the at least one stored acoustic input is defined as healthy, it may be seen desirable that the first acoustic input and/or the second acoustic input, which is compared with the at least one stored acoustic input, should be similar to the at least one stored acoustic input. Similar in this context may be understood as a difference between the respective compared acoustic inputs which falls below a respective threshold. The comparison of the first acoustic input and/or the second acoustic input with the stored acoustic input may be performed in analogy to the exemplary comparison of the first acoustic input with the second acoustic input as outlined above. Any deviation from e.g., the first acoustic input and/or the second acoustic input from the at least one other acoustic input may be interpreted as a change of the health state of the patient, such as e.g., a deterioration. The parameter-based definition of a deterioration of the health state of the patient may rest on the health care provider, the patient, etc. The (parameters of the) respective acoustic inputs and/or the threshold value(s) may be stored in a database, a blockchain and/or a cloud-based system. The storage in a blockchain may in particular provide the technical advantage of an increased data integrity and a decentralized data storage which may increase the associated data security. Each of the storage media may either be located on the system or may be located at a remote location (e.g., a computation center, a hospital information system, etc.).

As outlined above, the first and/or the second acoustic input may represent a wateriness in the lungs of the patient. If the first acoustic input is considered as representing a health state which is considered as “normal” (e.g., obtained directly after the discharge from a hospital) and the second acoustic input is obtained several weeks after the discharge (which e.g., does exhibit a sound pattern which may be related to the wateriness), it may be concluded, by the means for determining the deterioration, that a deterioration of the health state of the patient occurred. Additionally or alternatively, the health state which is considered as “normal” may also be represented by the at least one stored acoustic input and may thus act as a reference for what is considered as healthy.

Additionally or alternatively, if the acoustic input is a spoken sequence, the content of the spoken sequency may also be analyzed, e.g., by the means for determining (e.g., also a spoken sequence in response to a corresponding question provided by the system). This may facilitate the opportunity to evaluate how the patient uses words (e.g., whether repetitions are used, whether simple vocabulary is used, etc.) which may also act as a basis for comparing the second acoustic input with the first acoustic input and/or the at least one stored acoustic input and which may allow a conclusion whether the health state of the patient has deteriorated. In other words, if the patient suffers of e.g., a stroke which affects the speech center of the brain, the patient may tend to use a rather simple vocabulary. Simple vocabulary may e.g., be understood as avoiding technical terms, avoiding foreign language terms and/or a tendency to using simply structured sentences (e.g., by an absence of relative and/or long clauses). The content analysis of a spoken sequence may e.g., refer to the application of native language processing (NLP) which may allow a conversion of a spoken sequence to a text message which may further be processed (e.g., by analyzing the vocabulary as outlined above). Correspondingly, the means for determining may include means for NLP. However, also any other method for determining the content of a spoken sequence may be applicable which allows to convert an acoustic input (spoken sequence) into storable text data. It may further also be possible that the content of the spoken sequence is further processed. As an example, if the patient articulates that the patient suffers of persistent pain, said content-based articulation may also be considered by the means for determining.

Additionally or alternatively, the analysis of the content of the first and/or the second acoustic input may also allow conclusions about the subjective feeling of the patient. For example, the patient may be asked at least one question concerning the health state of the patient by e.g., a health care provider, a relative or any other empowered person. In response to the question, the patient may respond that she/he has the feeling that she/he suffers of dyspnea. Said content may additionally be used to determine whether the health state has deteriorated, e.g., by comparing the response of the patient to the same question which may also have been asked in the past.

The means for determining whether the health state of the patient has deteriorated may additionally or alternatively also comprise means for supplying a health care provider with the first acoustic input and/or second acoustic input. The health care provider may then compare the second with the first and/or at least one stored acoustic input of the patient. For example, a doctor may then (manually) determine whether a deterioration of the health state of the patient has occurred. Additionally or alternatively, it may also be possible that the received first acoustic input and/or the received second acoustic input are analyzed automatically. The first acoustic input and/or the second acoustic input may be transmitted to the health care provider as a sound stream (i.e., a recorded acoustic sequence) and/or as a transcript of the spoken sequence (i.e., after a conversion of the spoken sequence to a (storable) text).

The at least one stored acoustic input may relate to an acoustic input of the same patient which may have been recorded in the past and/or may relate to an acoustic input of at least one other patient which may have been recorded in the past.

The means for indicating a deterioration of the health state of the patient may relate to a visual and/or a haptic and/or an acoustic indication. A visual indication may exemplarily relate to a (blinking) light (e.g., by means of an LED, a flashlight, etc.) and/or a respective indication on a display (e.g., my means of a push notification). A haptic indication may relate to a vibration of e.g., a mobile phone and/or a smartwatch or any other suitable device. An acoustic indication may relate to an alarm, a spoken warning that the health state has deteriorated, etc. Alternatively or additionally, the means for indicating may also comprise an interface. The interface may relate to a communication interface which may facilitate a communication of the deterioration of the health state of the patient and/or a corresponding diagnosis to e.g., a telemetric center, e.g., a health care provider (e.g., by transmitting a respective message which indicates the deterioration and/or by establishing a telephone call to e.g., a health care provider), a hospital information system, e.g., an emergency unit which may quickly send an ambulance, a mobile and/or a smartphone, relatives of the patient, a wearable device (e.g., a smartwatch), a database, a smart home device (e.g., a smart speaker, a TV, etc.) or any other suitable device. In some examples, an automatic emergency call and/or request is initiated. In some examples, a phone call is automatically established with a health care provider, who may then, e.g., provide further instructions, e.g., to change medication. The communication may occur by means of a wired connection (e.g., ethernet, USB, etc.) or by means of a wireless connection (e.g., Wi-Fi, Bluetooth, RFID, NFC, 3G, 4G, 5G, etc.), including internet. Additionally or alternatively, it may also be possible that the patient is provided with at least one suggestion with respect to a medication or recommendations with respect to the avoidance of certain activities (e.g., the avoidance of sports) and/or the motivation for certain activities (e.g., related to the reduction of an alcohol consumption).

It may be possible that the first acoustic input and the second acoustic input are received automatically. Automatically may be understood that no dedicated/active action is required to receive the input, i.e., no request is required upon which the first acoustic input and the second acoustic input may be received.

However, the system may also comprise means for requesting the first acoustic input and/or the second acoustic input. For example, the actuation/pressing of e.g., a button (in hardware and/or software), and/or a predetermined schedule, may yield a request from the system to e.g., a (built-in) microphone (which may be unmuted as a result of the request), and/or it may cause the system to send a request to a user device (e.g., smartphone, smart home device, implant, etc. as outlined herein) that is to provide the acoustic input, and/or to e.g., a database system from which the acoustic input may then be provided. The first acoustic input and/or the second acoustic input may then be received by the system as outlined herein. In some examples, a request may be triggered, e.g., by authorized personnel, e.g., a doctor treating the patient and having access to the system via an interface, e.g., as described herein.

More specifically, if the system is implemented as a user device (e.g., a smartphone), the means for requesting a first acoustic input and/or a second acoustic input may send a corresponding request to e.g., at least one microphone of the user device. The at least one microphone may be part of the user device and the request may e.g., be understood as activating the microphone or, if the system is implemented as an app, as a request from the app to the microphone, i.e., to perform a recording and return the recording to the app. For example, one or microphones of the user device may automatically be activated subsequent to the request.

Additionally or alternatively, a request may be sent (by the system) to (another) user device such as e.g., a smartphone, a smart home device, a medical device, an implant, etc. and/or a server. In such a case the means for requesting acoustic input may comprise an interface as outlined above by which a request is sent to the external device. The first acoustic input and/or the second acoustic input may be recorded in response to the request by the external device (e.g., a microphone may be activated) and/or the external device may already have the first acoustic input and/or the second acoustic input stored and the request may simply initiate transmission to the system. The means for requesting may comprise means for transmitting a message-based request (e.g., a database query) and/or may comprise means for transmitting a remote procedure call (RPC)-like request. The RPC may e.g., start a process on a server which may repeatedly (as further described herein) request additional acoustic inputs (e.g., a third acoustic input, a fourth acoustic input, etc.).

Additionally or alternatively, a request for the first acoustic input and/or the second acoustic input may be provided to the patient, e.g., by means of a message on the display of the (external) user device, e.g., a smartphone (e.g., a push notification) and/or a smart home device (e.g., on a TV), and/or an acoustic message provided by the user device, etc. In response to the received request, the patient may activate the microphone on its own and/or the request may automatically activate the microphone wherein the message just has informative purposes.

The means for requesting may also comprise the means for asking the patient a question, e.g., about the current health state and/or any other generic question (e.g., concerning the present weather). It is also conceivable, that the question is provided by means of a telemetric connection (e.g., between a medical care provider having access to the system and the patient by means of a telephone call or any other suitable telemetric connection) with the patient. Moreover, it may also be possible that a medical care provider sends a (text-based) question to the user device (e.g., the smartphone) via the system, which may then appear as a push notification and which may be answered by the patient and which may be recorded by the built-in microphone of the user device.

Whether the system is implemented on a user device (e.g., on a smartphone) or a server, any communication related means may be based on a secure connection and/or encrypted. In any implementation, the system may foresee to ask the patient for permission prior to activating a recording. This may ensure the privacy of the patient at any time.

The means for requesting may further be adapted to base a request (for receiving the first acoustic input and/or the second acoustic input, or any other further acoustic input) on at least one of a predetermined schedule and/or an external input.

A predetermined schedule may e.g., be understood as certain (medical) check-up appointments. Said appointments may be defined by the patient and/or the health care provide or any other empowered entity (e.g., relatives, etc.). Said appointments may be entries into a (cloud-based) calendar according to which a reception of the first acoustic input and/or the second acoustic input and/or any additional acoustic input occurs. Said appointments may be defined periodically (e.g., every day, every week, every month, every year, etc.) or may be non-periodically by defining certain days on which the first and/or the second and/or any other acoustic input should be received. It may also be possible that the means for requesting are based on a timer function. As an example, if the patient changes a medication, the effect of the change of the medication on the health state of the patient may be seen as beneficial to be evaluated. In such a case, the health care provider or any other empowered entity may define a timer, such that e.g., after one hour of the last intake of the (changed) medication, a request for receiving an acoustic input may be provided to the system. The setting of the time may be based on a single action (i.e., a unique request) or may be self-renewing. In other words, the timer may also result in a periodic request for the reception of an acoustic input. Such a procedure may e.g., enhance the comfort of the patient as it is not required that the patient actively prompts the reception of the first acoustic input and/or the second acoustic input and/or any other acoustic input. This is in particular an important feature of the present disclosure as the determination of the deterioration of the health state of the patient may be seen as of particular relevance for people at a higher age who may tend to forget (medical) check-ups. Additionally, a beneficial effect of said feature may be seen in the aspect that no continuous reception of acoustic inputs needs to be activated. A continuous reception (and a potentially associated continuous recording of acoustic inputs) may be understood as a continuous surveillance of the patient which may be seen as a disturbing effect in view of the individual privacy of the respective patient. An initiated provision of acoustic data upon request only may therefore provide an increased privacy perception and no continuous surveillance of the patient.

The external input may be provided to the system by means of a wired (e.g. ethernet, USB, etc.) and/or a wireless communication (e.g. Wi-Fi, LTE, 5G, Bluetooth, RFID, NFC, etc.) of the system with at least one other device (e.g., a server, a mobile phone, a wearable, a medical device, a smart home device, etc. as outlined above). The external input may also be provided by a dedicated button and/or (mechanical) switch of the system and/or by a spoken command and/or the health care provider.

The system may further comprise means for receiving a value of at least one non-acoustic parameter associated with the health state of the patient. For a more concise determination of the deterioration of the health state of the patient or a further confirmation that the deterioration has indeed occurred, it may be possible to additionally receive a value of at least one non-acoustic input. Such a non-acoustic input may e.g., refer to at least one vital parameter of the patient. A value of a vital parameter may e.g., be understood as a blood pressure value, a pulse rate value, a blood oxygen value, a blood value or any other parameter which may be suitable to characterize the health state of the patient. Additionally or alternatively, the value of the at least one non-acoustic parameter may also refer to environmental parameters which characterize the surroundings of the patient. Such a value of a respective parameter may e.g., refer to a temperature value, a humidity value, GPS coordinates (i.e., to determine whether the current whereabouts of the patient is in a dry or a rather humidity saturated climate zone which may affect the circulation of the patient and thus the health state), etc. The value of the at least one non-acoustic parameter may be received by the system, e.g., via the internet (e.g., in a user device implementation, the user device may know the GPS coordinates and may retrieve the corresponding weather data from the internet). Additionally or alternatively, the value may be received from a dedicated sensor and/or device which may be part of the system (e.g., a sensor to measure the pulse rate or a blood oxygen concentration) and/or may be a separate device. The separate device may e.g., be a wearable (such as e.g., as smartwatch), a blood pressure gauge, an implant etc. An implant may e.g., be a pacemaker which may measure the current pulse rate and/or further heart related parameters. The system may receive the parameter via a suitable interface as described herein. Additionally or alternatively, the value of the at least one non-acoustic parameter may also be provided directly from a laboratory (e.g., relating to a blood value) and/or may also be obtained from a database (e.g., as part of a hospital information system and/or over the internet (e.g., weather information)). The value of the at least one non-acoustic parameter may relate to a quantitative parameter (as outline above) or may relate to qualitative parameters. Such qualitative parameters may e.g., relate to nutrition habits of the patient. In other words, the patient and/or the health care provider may provide the system with information concerning an alcohol consumption, consumed bread units (which may in particular be relevant for patients suffering of diabetes), information on fat food, etc.

The value of the at least one non-acoustic value may be received automatically (e.g., in addition to each of the acoustic inputs) and/or may be received upon an explicit request, e.g., by a request of the patient and/or the medical service provider and/or relatives of the patient. It is also possible that the value of the at least one non-acoustic input is requested by the system as it will further be described below.

The at least one non-acoustic input may also or additionally relate to 2^{nd} rank and/or 3^{rd} rank data. 2^{nd} rank data may be understood as data which is based on 1^{st} rank data (e.g., sensor data) but which has further been processed. For example, a blood oxygen concentration may be derived from a blood pressure and pulse rate measurement. In such a case, data may become available which is not directly accessible by a sensor. 3^{rd} rank data may be understood as 2^{nd} rank data which as further been processed and which may thus be understood as data with a higher level of abstraction as compared to 2^{nd} rank data and 1^{st} rank data.

The means for requesting the first acoustic input and/or the second acoustic input may be adapted to request the first acoustic input and/or the second acoustic input if the value exceeds a threshold. Upon the request, an acoustic input may be provided automatically, e.g., a microphone may be activated automatically, e.g., as described herein. Based on the received value of the at least one non-acoustic parameter it may be determined whether it may be useful to additionally receive at least the first acoustic input and/or the second acoustic input to concisely evaluate the current health state of the patient and to determine the potential deterioration of the health state of the patient. The at value of the at least one non-acoustic parameter may be received periodically and/or at pre-defined times (as described above) and/or upon an explicit request. The determination, whether the at least first acoustic input and second acoustic input should be received, may be based on a certain threshold. As an example, a medical care provider may define that a blood pressure which exceeds 120/80 mmHg is considered as harmful for the patient. If the value of the at least one non-acoustic parameter is e.g., received on a periodic basis, it may be possible to request the first acoustic input and/or the second acoustic input upon a comparison of the value of the at least one non-acoustic parameter (e.g., the blood pressure) with the threshold (e.g., 120/80 mmHg) which may be seen as healthy. This may in particular facilitate a situational reaction on the health state of the patient and may facilitate an early determination whether the health state of the patient has deteriorated. Moreover, this also allows to minimize computational load and optimize privacy issues during which the value of the at least one non-acoustic parameter is below the threshold value. For the sake of completeness, it is further noted that the means for requesting the first acoustic input and/or the second acoustic input is not limited to the parameter “blood pressure” but may also be applied to any other parameter which is suitable to be associated to the health state of the patient, such as e.g., any blood value, the heart rate, etc., and which may be applicable for preemptive care taking.

In an exemplary embodiment, the system for determining a deterioration of a health state of a patient comprises a) means for receiving a first acoustic input, associated with the health state of the patient at a first time, and a second acoustic input, associated with the health state of the patient at a second time; b) means for determining whether the health state of the patient has deteriorated based at least in part on a comparison of the first acoustic input with the second acoustic input; and c) means for indicating the deterioration of the health state of the patient, if it is determined that the health state of the patient has deteriorated. The system further comprises means for requesting the first acoustic input and/or the second acoustic input and means for receiving a value of at least one non-acoustic parameter associated with the health state of the patient. Furthermore, the means for requesting the first acoustic input and/or the second acoustic input is adapted to request the first acoustic input and/or the second acoustic input if the value exceeds a threshold.

The features contained in this embodiment (above) make it possible to simplify a situational reaction to the state of health of the patient, which enables an early determination, whether the health status of the patient has deteriorated. In addition, the computing load can be minimized, and privacy can be optimized if the value of the at least one non-acoustic parameter is below the threshold.

Additionally or alternatively, it may also be possible that at least a third acoustic input is requested in response to the value of the at least one non-acoustic parameter exceeding a threshold. As an example, it may be possible that the first acoustic input and the second acoustic input have safely been received by the system and the health state of the patient has been diagnosed as healthy (i.e., non-deteriorated) by the means for determining according to the present disclosure. However, in some situational contexts, this may not be the case, and/or the data indicate that it may be likely that the health state of the patient will not remain in a satisfying (i.e., a healthy) state over time. Hence, an additional at least one third acoustic input may be requested for a further concise characterization of the current health state of the patient. This approach essentially follows the idea to base the determination, whether the health state of the patient has deteriorated, on a larger data basis (i.e., more values of parameter and/or parameters, associated with the health state of the patient should be provided to the means for determining).

The means for receiving the first acoustic input and/or the second acoustic input may comprise means for receiving a voice input and/or a respiratory sound input. The first acoustic input and/or the second acoustic input may e.g., relate to a voice input which may preferably be (but not limited to) a spoken sequence. The spoken sequence may e.g., be an arbitrary sequence of words spoken by the patient and/or may be a dedicated response to a question which was directed to the patient beforehand. Additionally or alternatively, the voice input may also relate to a singing and/or humming of the patient. In addition or alternatively, the first acoustic input and/or the second acoustic input may also be a respiratory sound. This may generically relate to any sound which is associated with the respiratory system of the patient. As an example, this may relate to a breathing sound, a cough sound, etc. As an example, if the patient suffers of a cardiac weakness or myocardial failure, it is known that patients tend to suffer from wateriness, in particular, in the lungs. Water in the lungs may e.g., lead to a rales-like sound during breathing which may be considered as a typical indicator for a deterioration of the health state of the patient if an accumulation of water occurs. If the system according to the present invention further determines the deterioration of the health state of the patient, this may be indicated early on. In addition, an associated indication for the deterioration may be provided to the health care provider. Based thereon the medical care provider may quickly administer drugs which may remove the water from the lungs. Requesting/receiving an additional third acoustic input may then be used to evaluate the effectiveness and the success of the medication.

The system may further comprise means for generating a patient-specific profile based at least in part on the first acoustic input and the second acoustic input. Additionally, the means for determining may be adapted to determine whether the health state of the patient has deteriorated based at least in part on a comparison of the generated patient-specific profile with at least one stored patient-specific profile.

It may be possible that the first acoustic input and the second acoustic input are combined to a patient-specific profile. The patient-specific profile may be understood, e.g., as a flashcard which may be unambiguously assignable to the patient, e.g., by means of an identification number (which may also be provided in the patient-specific profile) which (anonymously) identifies the patient.

The patient-specific profile may additionally also be provided with the value of the at least one non-acoustic parameter (as described above). The means for generating the patient specific-profile may hence further comprise means for including the value of the at least one non-acoustic parameter in the profile. It may also be possible that more than one acoustic input and more than one non-acoustic input is stored in the generated patient-specific profile. It may be possible to store the raw data of the received first acoustic input and/or the second acoustic input in the generated patient-specific profile. Additionally or alternatively, it may also be possible to store the first acoustic input and/or the second acoustic input at a higher level of abstraction in the profile. A higher level of abstraction may be understood as a parameter which is derivable from the raw data associated with the first acoustic input and/or the second acoustic input. For the sake of clarity, such derivable parameters may e.g., be a pitch of a voice of the patient, speed of speaking, etc.

It may further be possible to request at least one further acoustic input and/or at least one further non-acoustic input in case no stored patient-specific profile can be found among the at least one stored patient-specific profile. In such a case, the at least one further acoustic input and/or the at least one further non-acoustic input may be used to increase the provided data basis and/or may be used to explain the discrepancy. E.g., if existing data motivates the conclusion that the patient (spontaneously) suffers from breathlessness (which cannot be explained on the basis of the stored patient-specific profiles), at least one further non-acoustic parameter may be requested, e.g., a GPS location of the patient. As a result, it may turn out that the patient is currently visiting a high humidity climate zone. It is known that such climate conditions may negatively affect the health state of a patient. Based on this additional information, the determination, whether the health state of the patient has deteriorated, may be repeated.

It may be possible that there is one unique patient-specific profile per patient wherein a plurality of acoustic inputs and/or a plurality of values of non-acoustic inputs may be stored. Such a plurality of inputs may e.g., facilitate the tracking of a time evolution of the respective inputs and may facilitate a monitoring of the health state of the patient (and in particular a potential deterioration of the health state of the patient) over time. Preferably, the stored acoustic inputs and/or non-acoustic inputs are provided with a time stamp (as outlined above) which may allow a replicability of a potential deterioration of the health state of the patient (with respect to the temporal evolution). Notably, a time stamp may facilitate the determination of the temporal spot at which the deterioration of the health state of the patient started/occurred and it may additionally be possible to determine how quickly (e.g., by means of the calculation of a (time) derivative) the health state of the patient may have deteriorated. This information may be used do adjust the administration of pharmaceuticals accordingly. Alternatively, it may also be possible that a new patient-specific profile is generated at each time at which a first acoustic input and a second acoustic input (and optionally additional acoustic inputs) is received. In such a scenario, a generated patient-specific profile may be understood as a momentaneous capture/snapshot of the health state of the patient.

The generated patient-specific profile may additionally be provided with qualitative data which may be associated with the health state of the patient, e.g., age, weight, allergies, is nutrition habits, etc.

The generated patient-specific profile may be stored temporarily (e.g., in a transient and/or a non-transient memory) or may be stored permanently (e.g., in a database, a blockchain, a cloud-based system, etc.). The generated patient-specific profile may be stored locally (e.g., in the system according to the present disclosure) and/or may be stored remotely (e.g., in a computation center, a hospital information system, a cloud-based system etc.).

Furthermore, it may be possible that the at least one stored acoustic input is also associated with a respective at least one stored patient-specific profile. The stored patient-specific profile may either be assigned to the same patient and/or to any other patient who may have a similar medical record (e.g., similar age, similar weight, similar allergies, similar pre-existing diseases, etc.). Similar to the generated patient-specific profile, as discussed above, it may be possible that the stored patient-specific profile comprises a first acoustic input and/or a second acoustic input and may additionally also comprise at least one value of the at least one non-acoustic parameter. Moreover, it is possible that the at least one stored patient-specific profile comprises more than the first acoustic input and/or the second acoustic input (and optionally more than one non-acoustic input, i.e., a plurality of non-acoustic inputs). Said additional inputs may relate to inputs which were recorded/received at different times and may thus (similarly as outlined above) allow a tracking/monitoring of the health state of the patient over time. The stored at least one patient-specific profile may therefore also be understood as a historic data set which at least partially mirrors the medical record of the respective patient.

Additionally, it may also be possible that the at least one stored patient-specific profile is assigned with a positivity value which may be understood as a score value indicating the health state of the patient (which may be encoded by the first acoustic input and/or the second acoustic input). In other words, the positivity value may provide the option to “grade” the health state of the patient within a certain grading range. The grading range may e.g., span grades between 0 (e.g., an alarming health state of the patient) and 10 (e.g., a perfect/desirable health state of the patient). However, said grading range is only mentioned exemplarily and any other suitable grading scheme may also be possible. The grading may e.g., be assigned by the patient (e.g., based on a subjective perception) and/or may be based on a perception of the health care provider (e.g., based on an anamnesis, laboratory parameters, experience, etc.).

The means for comparing the generated patient-specific profile with the at least one stored patient-specific profile may be understood as means for a comparison of the first acoustic input and/or the second acoustic input with each other and/or with a respective stored first acoustic input and/or a stored second acoustic input which is included in the at least one stored patient-specific profile. Based on said comparison, it may be determined by how much the first acoustic input and/or the second acoustic input has deviated from the respective stored first acoustic input and/or the respective stored second acoustic input. The deviation, by how much two evaluated acoustic or non-acoustic inputs deviate from each other, may e.g., be expressed by the calculation of at least on metric among the respective pairs of acoustic inputs (more specifically, the derivable parameters). Such a metric may, in its simplest form, be implemented as the calculation of the difference of two values of the respective parameter. However, it is noted that the calculation of a difference may be understood as an example only. More complex approaches (e.g., by including additional weighting factors) may equally be applicable. Said weighting factors may facilitate to emphasize the contribution of certain parameters in the generated patient-specific profile and the at least one stored patient-specific profile (and their relevance for the health state of the patient). If the deviation exceeds a threshold value, the situation may be understood as a deterioration of the health state of the patient (as outlined above). Additionally, it may also be possible to include the at least one non-acoustic parameter when determining whether the health state of the patient has deteriorated. This may be done by comparing the respective values of the non-acoustic parameter (as obtained) with the respective stored value. The determination of the deviation may also be understood as the calculation of a metric (i.e., the difference of respective values which may be used to make a conclusion with respect to the health state of the patient, as outlined above). If the calculated metric is minimized for a certain stored patient-specific profile, said profile may be understood as most similar to the generated patient-specific profile. Moreover, it may also be concluded that said profile relates to a (historic) health state of the same or another patient which may be considered similar to the current health state of the patient. Therefore, if the (historic) health state of the patient had been scored as “healthy” or “normal”, the current health state may also be considered as “healthy” or “normal”. As outlined above, the most similar stored patient-specific profile may also be assigned with a positivity value. In such a case, the current first acoustic input and/or the current second acoustic input may be associated with an evaluation whether the current health state is alarming or may considered as satisfying/normal.

The means for generating the patient-specific profile may further comprise means for including the value of the at least one non-acoustic parameter in the profile. As it has briefly been outlined above, it may also be possible to store the at least one non-acoustic input in the generated patient-specific profile. The value of the at least one non-acoustic parameter may additionally be assigned with a time stamp for a replicability of the respective time evolution as it has been outlined above. Including the non-acoustic profile in the generated patient-specific profile may allow for a redundancy check whether a deterioration of the health state of the patient has indeed occurred. Whereas the initial determination of a deterioration of the health state of the patient may be based on the first acoustic input and/or the second acoustic input, the at least non-acoustic parameter may additionally be involved to confirm the initially determined deterioration. For example, if the comparison of the first acoustic input with the second acoustic input and/or with the stored first acoustic input and/or with the stored second acoustic input has led to the conclusion that the health state of the patient has deteriorated, the value of at least one non-acoustic value may additionally be considered. If said value relates to a blood pressure which is not contained in an interval which is considered as healthy, the initially determined deterioration of the health state of the patient may be understood as confirmed.

Additionally or alternatively, the at least one non-acoustic input may also be comprised by the respective stored patient-specific profiles. In such a case, the non-acoustic profiles may also be part of the comparison of the generated patient-specific profile with the at least one stored patient-specific profile. In a further example, the respective values of the at least one non-acoustic parameter may also be involved when calculating the at least one metric between the generated patient-specific profile and the at least one stored patient-specific profile.

Additionally or alternatively, the system may provide the first and/or second acoustic input, optionally the non-acoustic input, and/or the patient-specific profile to a hospital information system.

The system may further comprise means for providing the patient with a request for at least one additional acoustic input if a deterioration of the health state of the patient is determined. The request may comprise at least one question, as explained above. In addition or alternatively to the above-mentioned inclusion of the at least non-acoustic parameter, to evaluate whether the health state of the patient has indeed deteriorated, it may also be possible to provide the patient with an additional or alternative request. The request may e.g., be a question, asked by the system, by the medical care provider, a relative or any other empowered entity. The question may either be directed to the health state of the patient or may be of a generic type (e.g., a question about the current weather situation) with the goal to receive at least one more acoustic input from the patient. Therefore, the amount of data which may be considered for the (final) determination whether the health state of the patient has deteriorated may be increased and the conclusion may be understood as more reliable.

The means for providing the patient with the request for at least one additional acoustic input may be understood in analogy to the means for requesting the first acoustic input and/or the second acoustic input and may be based on the same concepts as outlined above.

Moreover, the patient may also be provided with a request for at least one additional acoustic input if no stored acoustic input is found among the at least one stored acoustic input (or among the at least one stored patient-specific profile) which is considered as similar to the first acoustic input and/or the second acoustic input. In such a case, it may be seen beneficial to increase the respective data basis by requesting at least one further acoustic input and/or one further non-acoustic input. The determination whether the health state of the patient has deteriorated may then be performed again on the basis of the increased data basis.

Similarly as outlined above, the additional acoustic input may also be stored to the optional generated patient-specific profile and/or may be provided with a time stamp.

The first acoustic input and/or the second acoustic input and/or the other (stored) acoustic input may additionally be associated with a positivity value. As it has already briefly been outlined above (in conjunction with the generation of a patient-specific profile), also the first acoustic input and/or the second acoustic input may be provided with a positivity value to evaluate the health state associated with the respective acoustic value (as it has been outlined above in analogy to the generated patient-specific profile). A grading system similar to the grading system as outlined above may be used for that purpose. The positivity value may be supplied by the patient, the medical care provider, relatives, etc. as it has also been outlined above. The positivity value may be associated with the received first acoustic input and/or the received second acoustic input and/or may also be (historically) associated with the at least one stored acoustic input. When comparing the received first acoustic input with the received second acoustic input and/or with the at least one stored acoustic input, a stored acoustic input may be determined first which is considered as most similar to the received first acoustic input and/or the received second acoustic input. The determination of the most similar stored acoustic input may also be based on a determination of at least one metric between the received acoustic inputs and the at least one stored acoustic inputs. The stored acoustic input for which the at least one metric may be minimized may then be regarded as most similar to the current acoustic input which may be represented by the received first acoustic input and/or the received second acoustic input. If the determined most similar stored acoustic input is then provided with a positivity value which indicates a certain health state of the patient (e.g., considered as healthy), also the received first acoustic input and/or the received second acoustic input may be considered to be associated with that health state of the patient.

The system may further comprise means for analyzing the first acoustic input and/or the second acoustic input and/or the additional acoustic input and wherein the means for analyzing comprises means for determining a pitch of the a voice of the patient and/or a speech disruption and/or slow speaking and/or a vocal tremor and/or at least one repetition during speaking and/or a breathlessness, and/or means for converting the acoustic input into a storable text message.

The reception of the first acoustic input and/or the second acoustic input and/or any additional acoustic input may be followed by a deduction of at least one parameter which may be used to make a conclusion whether the health state of the patient has deteriorated. Such parameters may, e.g., be the pitch of the voice of the patient. The pitch of the voice may be understood as the absolute frequency of the voice, i.e., if the absolute frequency of the voice is shifted towards higher frequencies (in the acoustic spectrum), the voice may be experienced as rather high. If the absolute frequency of the voice is shifted towards lower frequencies, the voice may be experienced as a rather low voice. The determination whether the pitch of the voice of the patient has changed may be based on a variety of options. It may be possible that the acoustic frequency spectrum is divided into frequency chunks of a certain frequency bandwidth (e.g., 50 kHz). The spoken sequence of the patient (which may be understood as the first acoustic input) may undergo a Fourier transformation to obtain the individual frequency components of the voice. Additionally, when considering the result of the Fourier transformation (preferably a fast Fourier transform (FFT)) in e.g., a power vs. frequency spectrum (e.g., a power spectral density-(PSD-) diagram), the area between the Fourier spectrum and the frequency axis may then be calculated for each of the frequency chunks. Said area may be understood as the acoustic spectral power in each of the chunks. The chunk which may result in the highest/maximized acoustic power may be regarded as the dominating chunk for the determination of the pitch of the voice. If e.g., the chunk with the maximum power is at 5 kHz (first acoustic input) and shifts towards 6 kHz (second acoustic input) over time, this shift may be considered as related to a deterioration of the health state of the patient as it may e.g., be caused by dyspnea and/or symptoms of a stroke.

Another acoustic parameter which may beneficially be used to determine a health state of to the patient may be based on the analysis of speech disruptions. Speech disruptions may be understood as intended or unintended breaks while speaking. Said breaks may occur due to breathlessness of the patient and/or due to neuronal focus-based disruptions as a result of e.g., a neuronal disease. In such a scenario, the patient may tend to include more breaks while speaking in e.g., a first acoustic input and/or a second acoustic input as compared to e.g., at least one stored (historic) acoustic input (associated with a satisfying health state of the patient). Such characteristic changes, while speaking, may also be used to determine the current (or a deterioration of the) health state of the patient. A similar conclusion (with respect to the health state of the patient) may be based on an analysis of the speaking speed of the patient. This may e.g., be expressed by the number of words spoken per minute as it may exemplarily be analyzed by a natural language processing (NLP) algorithm (also other applicable algorithms may be possible). An NLP algorithm may transform the spoken sequence into a series of words (i.e., a written text which may be stored) which may also be associated with a time axis such that the number of spoken words per second may be counted. If the number of spoken words is decreased in the second acoustic input as compared to the first acoustic input and/or as compared to the at least one stored acoustic input (which may e.g., be regarded as similar to the current acoustic inputs), this may be interpreted as an indication for neuronal focusing-related aspects and/or breathlessness. On the other hand, it may also be possible that the patient shows an increased speed while talking, i.e., the patient articulates an increased number of words as compared to the (historic) reference acoustic input. In such a case, the current health state of the patient may e.g., be interpreted as nervous and/or anxious which may be associated with other neuronal diseases.

A further indicator for the health state of the patient, which may also (or alternatively) deviate between acoustic inputs, is the potential existence of a vocal tremor. A vocal tremor may be understood as a vibrating voice while speaking. Such vibrations may e.g., be derived from an analysis of the time series of a spoken sequence and/or an FFT to extract the respective frequencies. If said vocal tremor occurs suddenly, e.g., between the respective recording of the first acoustic input and the second acoustic input and/or the at least one stored acoustic input, it may also be used as an indicator for a determination of the health state of the patient.

Another parameter which may be derived from a spoken sequence of the patient (as being part of the first acoustic input and/or the second acoustic input and/or any other acoustic input) is the number of repetitions. Said repetitions may e.g., refer to the amount of content repetitions. For example, if a patient suffers from a weakness of memory, the patient may is talk about the same subject repeatedly, potentially without being aware of the fact that the same subject matter had been addressed beforehand.

It is further noted that said potential parameters, derivable from the first acoustic input and/or the second acoustic input are only mentioned exemplarily and that any other parameter which may also allow a conclusion from the respective acoustic input to the health state of the patient may also be suitable. However, the specific derivable parameters, as discussed herein, may in particular provide the advantage that an acoustic input may be transformed into at least one quantitative parameter which simplifies the determination of temporal changes of said parameters which are essentially associated with the health state of the patient. Any deterioration of the health state of the patient may thus more clearly be determined. For the sake of completeness, it is noted that the same procedure may also be applied to any further acoustic input (i.e., a third acoustic input, a fourth acoustic input, etc.).

The present disclosure further relates to a system for determining a deterioration of a health state of a patient wherein the system comprises means for transmitting a first acoustic input, associated with the health state of the patient at a first time, and a second acoustic input, associated with the health state of the patient at a second time, (to an apparatus) for determining whether the health state of the patient has deteriorated, based at least in part on a comparison of the first acoustic input with the second acoustic input. Additionally, the system may comprise means for receiving an indication of the deterioration of the health state of the patient in response to transmitting the first and second acoustic input, if, based at least in part on the comparison, it is determined (by the apparatus) that the health state of the patient has deteriorated.

In some examples, the determination may additionally or alternatively be based at least in part on a comparison of the first acoustic input with at least one other acoustic input.

Similarly as outlined above, the means for transmitting and receiving may comprise an interface wherein the interface may be a software or a hardware interface. If implemented in software, the interface may relate to a certain port by means of which the transmission is may occur. Alternatively it may also be possible that the interface is implemented as a (web) socket. If implemented in hardware, the interface may facilitate both a wired (e.g., ethernet, USB, etc.) and/or a wireless communication (e.g., Wi-Fi, Bluetooth, RFID, etc.).

The system may be implemented as a mobile device (such as e.g., a mobile phone, a wearable (e.g., a smartwatch), a medical device (e.g., a stationary or dedicated wearable device, an implant), etc. The system may be implemented as a (standalone) device (as discussed beforehand) or may be part of a device (e.g., the system may be an app and/or an integrated circuit, e.g., of a smartphone, a patient device, etc.). A dedicated wearable device may e.g., refer to a device which is designed to determine a deterioration of the health state of the patient. Such a device may comprise a microphone, processing electronics (e.g., at least one of a processor, a transient and/or non-transient memory, a bus system, and/or means for indicating a potential deterioration of the health state of the patient).

If implemented as a user device, such as a smartphone, the system may e.g., receive the first acoustic input and/or the second acoustic input from a microphone of the smartphone, from a smartwatch, a smart home device or any other suitable device which may be used to provide an acoustic input to the exemplary smartphone. The smartphone may then transmit the first acoustic input and/or the second acoustic input to a server-based system such as e.g., a cloud-based system which may comprise an AI for the determination whether the transmitted (by the smartphone) first acoustic input and/or the second acoustic input, possibly in combination with other acoustic data, may encode a deterioration of the health state of the patient. The other acoustic data may be stored data as outlined herein. The result of the comparison (and thus also of the determination) may be transmitted to the smartphone and received (by the smartphone) as an indication that the health state of the patient has deteriorated in case a deterioration had been determined beforehand in the cloud-based system. The indication may then be an alarm (or any other indication as it has been outlined above) to alert the patient and/or the health care provider. Additionally or alternatively, the indication may also be associated with a suggestion for a medication to return the health state of the patient to a health state which is considered as healthy.

In other examples, a corresponding AI may be implemented at least in part in the user device itself. The user device may then only leave parts of the determination to the cloud-based system, or none at all. In the former case, the user device may perform a preliminary assessment of the acoustic and/or non-acoustic input. The results of this preliminary analysis may be forwarded to the cloud-based system which may then process the corresponding (preliminarily analyzed acoustic and/or non-acoustic input). In the latter case, the user device may use the cloud-based system as a storage medium only.

Additionally, the present disclosure relates to a method for determining a deterioration of a health state of a patient wherein the method may comprise receiving a first acoustic input, associated with the health state of the patient at a first time, and a second acoustic input, associated with the health state of the patient at a second time. Additionally, the method may relate to determining whether the health state of the patient has deteriorated based at least in part on a comparison of the first acoustic input with the second acoustic input. Additionally, the method may comprise indicating the deterioration of the health state of the patient, if it is determined that the health state of the patient has deteriorated.

Moreover, the present disclosure relates to a method for determining a deterioration of a health state of a patient wherein the method may comprise transmitting a first acoustic input, associated with the health state of the patient at a first time, and a second acoustic input, associated with the health state of the patient at a second time, (to an apparatus) for determining whether the health state of the patient has deteriorated, based at least in part on a comparison of the first acoustic input with the second acoustic input and/or with at least one other acoustic input. Additionally, the method may comprise receiving an indication of the deterioration of the health state of the patient in response to transmitting the first and second acoustic input, if, based at least in part on the comparison, it is determined (by the apparatus) that the health state of the patient has deteriorated.

Additionally or alternatively, the determination whether the health state of the patient has deteriorated may also be based at least in part on a comparison of the first acoustic input with at least one other acoustic input.

The present disclosure further comprises a computer program which may comprise code which may cause a computer to implement the method steps described herein, and/or the means as described herein, when the instructions are executed.

Whether described as method steps, computer program and/or means, the functions described herein may be implemented in hardware, computer programs, software, firmware, and/or combinations thereof. If implemented in software/firmware, the functions may be stored on or transmitted as one or more instructions or code on a computer-readable medium. Computer-readable media includes both computer storage media and communication media including any medium that facilitates transfer of a computer program from one place to another. A storage medium may be any available media that can be accessed by a general purpose or special purpose computer. By way of example, and not limitation, such computer-readable storage media can comprise RAM, ROM, EEPROM, FPGA, CD/DVD or other optical disk storage, magnetic disk storage or other magnetic storage devices, or any other medium that can be used to carry or store desired program code means in the form of instructions or data structures and that can be accessed by a general-purpose or special-purpose computer, or a general-purpose or special-purpose processor.

It is further noted that the present invention is not limited to the specific feature combinations expressly listed herein, which are only understood as examples. Other features and/or feature combinations may also be possible.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

The following figure is provided to facilitate the understanding of the present invention:

FIG. 1 Illustration of a flow chart of a possible implementation of the medical voice bot system/method according to the present disclosure.

### DETAILED DESCRIPTION

The following detailed description of the present invention and further embodiments described therein are provided to facilitate the understanding of the present invention.

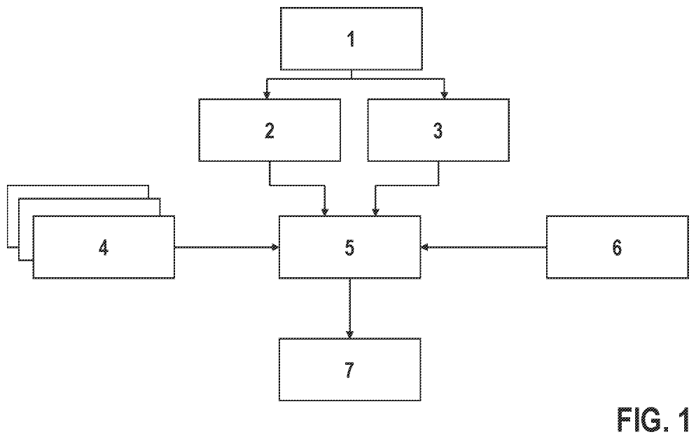

FIG. 1 shows a flow diagram of an exemplary embodiment of the system according to the present invention. The determination of the deterioration, according to FIG. 1, may be performed in a system which may e.g., be embodied as a smartphone, a smart home device (e.g., a smart speaker, smart TV, etc.), a wearable (e.g., a smartwatch), etc. Alternatively, it may also be possible that the system may be embedded as part of a server-side service. Further details on the embodiment will be described below.

The initialization of the system and thus the determination whether the health state of the patient has deteriorated may be based on an optional (initial) request 1 for a first acoustic input 2 and/or a second acoustic input 3.

The request 1 may be initiated by the patient, a medical service provider (e.g., a doctor, a nurse, etc.) and/or any other authorized person. It may be possible that the request 1 is directly initiated by means of a switch and/or a touch sensitive button/screen (e.g., if the system is embodied as a respective user device). Moreover, it may also be possible that the request is initiated acoustically, e.g., by a spoken sequence which may be captured, e.g., by a smart home device, and then e.g., forwarded to the system. The request may also be based on a predetermined schedule as outlined herein. The request may also be initiated remotely, e.g., by a doctor, e.g., by transmitting a corresponding message to the system.

The system according to the present invention may provide the request and, in response to the request, e.g., at least one microphone may be activated (e.g., which is in connection with the system, e.g., as being integrated into the system (if the system is exemplarily embodied as a smartphone), and/or in wireless or in wired connection with the system). An activation of the at least one microphone may e.g., be understood as unmuting the microphone and/or as establishing a connection to the microphone which may allow for a data transmission from the microphone to the system (e.g., a web socket connection, a Bluetooth connection, or a simple internal connection within a smartphone etc.). Additionally or alternatively, the request may be provided to a database and/or a separate user device, from which the first acoustic input 2 and/or the second acoustic input 3 may be received. In other words, the first acoustic input and/or the second acoustic input may also already exist in e.g., a database and/or may be generated, i.e., recorded, as a result of requesting the respective input (e.g., by activating the at least one microphone or any other suitable device for supplying the respective input). If the system is implemented as a server, the server may send the request as a message to e.g., a database which may then return (in response) the respective first acoustic input 2 and/or the second acoustic input 3. Additionally or alternatively, the server may also send the request, as e.g., a remote procedure call (RPC), to start a remote procedure on another device, e.g., a smart home device, a smartphone, etc., which may initiate a recording of the first acoustic input 2 and/or the second acoustic input 3.

The first acoustic input 2 and the second acoustic input 3 may have been created at different times, such that e.g., the second acoustic input 3 may have been recorded after the recording of the first acoustic input 2. However, also any other temporal separation between the first acoustic input 2 and/or the second acoustic input 3 may be possible. It may also be possible that the first acoustic input 2 and/or the second acoustic input 3 are provided with a timestamp.

As a consequence of requesting the first acoustic input 2 and/or the second acoustic input 3, the respective inputs may be received at the system (according to the present disclosure) for further processing. Prior to proceeding, the first acoustic input 2 and/or the second acoustic input 3 may be stored temporarily, i.e., in transient storage medium (e.g., a flash memory) and/or in a non-transient storage medium (e.g., a hard drive, a database (as part of the system or located in a remote computation center)).

However, it may also be possible, that the first acoustic input 2 and/or the second acoustic input 3 are received without a dedicated request. In such a case, the respective at least one microphone, the respective database, etc. may, for example, periodically (as outlined above) transmit an acoustic input to the system. Additionally or alternatively, it may also be possible that the system receives the first acoustic input and/or the second acoustic input based on any other suitable time base (e.g., based on a calendar entry, as outlined above).

The received first acoustic input 2 and/or the second acoustic input 3 may be supplied to means for determining 5 whether the health state of the patient has deteriorated or not. The means for determining 5 may be based on a comparison between the first acoustic input 2 and the second acoustic input 3. In case the second acoustic input 3 has been recorded after the recording of the first acoustic input 2, the second acoustic input 3 may be understood as referring to a health state of the patient at a time subsequent to the time at which the first acoustic input 2 has been recorded.

Additionally or alternatively, the means for determining 5 may also comprise means for comparing the first acoustic input 2 and/or the second acoustic input 3 with at least one stored acoustic input 4. The at least one stored acoustic input 4 may be an acoustic input which has been recorded e.g., one year before the present evaluation of the health state of the patient (or at any other time in the past).

Similarly as outlined above, the first acoustic input 2 and/or the second acoustic input 3 may be compared to the at least one stored acoustic input 4. Therefore, if the first acoustic input 2 and/or the second acoustic input 3 were recorded after the at least one stored acoustic input 4, it may also be possible to deduce a temporal evolution of the health state of the patient from said comparison.

The comparison (as performed by the means for determining 5) itself may be manifold and may be carried out locally in a user device or carried out remotely (e.g., on a server). If carried out in a user device, the means for determining 5 may be part of an app. If carried out remotely, the means for determining 5 may be understood as a SaaS, HaaS, IaaS based system, more specifically a cloud.

It may be possible that the at least one stored acoustic input 4 possesses a positivity value which may be understood as an indication for the extent to which the patient has been regarded as healthy when the associated stored acoustic input 4 has originally been obtained (e.g., by recording). If the comparison of the first acoustic input 2 and/or the second acoustic input 3 shows a similarity with one of the at least one stored acoustic input 4, the present health state of the patient may be considered as similar to the health state when the at least one stored acoustic input 4 was recorded. If e.g., the at least one stored acoustic input 4 is associated with a satisfying health state and the first acoustic input 2 and/or the second acoustic input 3 shows a discrepancy with respect to the at least one stored acoustic input 4, the health state of the patient may be considered as deteriorated. The discrepancy may e.g., be expressed as the calculation of at least one metric between the at least one stored acoustic input 4 and the first acoustic input 2 and/or the second acoustic input 3. The at least one metric may, in its simplest embodiment, be understood as a difference between the first acoustic input and the second acoustic input (other approaches are also possible as outlined above).

Since the first acoustic input 2 and/or the second acoustic input 3 may relate to e.g., a spoken sequence which may only be seen as a qualitative feature, a variety of parameters, describing the acoustic input, may be derived from said inputs. As it has been outlined above, by a further processing (e.g., a FFT, a NLP, etc.) of the first acoustic input 2 and/or the second acoustic input 3, it may be possible to derive quantitative parameters (e.g., words spoken per minute, pitch of the voice of the patient, etc. and as described above) which may be used for the calculation of the at least one metric between the at least one stored acoustic input and the first acoustic input 2 and/or the second acoustic input 3.

Additionally or alternatively, it may also be possible to generate a patient-specific profile based on the first acoustic input 2 and/or the second acoustic input 3. The generated patient-specific profile may be understood in analogy to a flashcard which comprises at least the first acoustic input 2 and/or the second acoustic input 3. Moreover, the flashcard may also comprise a patient-specific identification number which may be used to unambiguously identify the patient. It may also be possible that the generated patient-specific profile comprises at least one further acoustic input. Such a plurality of acoustic inputs (of the same patient), which were ideally recorded at different times, may facilitate the tracking of the evolution of the health state of the patient. Preferably, each of the acoustic inputs of the plurality of acoustic inputs may be provided with a time stamp. It may further be possible to store the plurality of acoustic inputs in a single generated patient-specific profile and it may be possible that a plurality of patient-specific profiles are generated wherein each of the profiles comprises one or more acoustic inputs (each Optionally be associated with a certain time).

The generated patient-specific profile (or the plurality of patient-specific profiles) may be stored locally in the system (e.g., in a dedicated database and/or a blockchain and/or any other suitable data management system) and/or remotely in a cloud, a blockchain, a server wherein the remote storage may occur in a computation and/or data center which may be accessible over the internet or locally through a local area network.

The means for determining 5 may also foresee means for comparing the generated patient-specific profile (or the plurality of generated patient-specific profiles) to at least one stored patient specific profile. For example, a stored profile may comprise acoustic and/or non-acoustic inputs and/or one or more parameters pertaining to such inputs. When comparing two profiles, at least one parameter (as outlined herein), derivable from and associated with the acoustic inputs, stored in the profile, may be used for the calculation of at least one metric (wherein the metric may possess weighting factors to emphasize and/or to account for the specific relevance of certain parameters). The goal of the comparison may be seen to seek for a stored patient-specific profile for which the metric is minimized. Such a profile may then be understood as most similar to the present generated patient-specific profile (or the respective plurality of patient-specific profiles).

In case a deterioration of the health state of the patient is determined, it may be desirable to is re-evaluate the determination that the health state of the patient has deteriorated, based at least in part on at least one further acoustic input and/or at least one non-acoustic input 6 (e.g., a vital parameter of the patient as outlined above). This may allow for a confirmation of the deterioration of the health state prior to indicating the deterioration to the patient.

An additional at least one acoustic input may be provided to the system as a result of the determination of the deterioration of the health state of the patient. Said additional at least one acoustic input may be a vocal input by the patient, such as e.g., a spoken sequence by the patient and/or may be a respiratory sound (e.g., a sound associated with the breathing of the patient). As it has been mentioned above, each of said acoustic inputs may be used to derive at least one parameter which may be associated with the health state of the patient. The additional at least one acoustic input, if embodied as a spoken sequence, may be received from at least one microphone and/or received from at least one database, a blockchain and/or a cloud. The microphone may be part of a smartphone, may be a standalone microphone and/or may be part of a smart home device such as a smart speaker, a smart TV, etc. The database, blockchain and/or cloud may be part of the system or may be arranged remotely (e.g., in a computation center) as also outlined above.

Also if the means for determining 5, whether the health state of the patient has deteriorated, cannot find an acoustic input, among the at least one stored acoustic inputs or the at least one stored patient-specific profile respectively, which may be considered as similar to the present first acoustic input 2 and the second acoustic input 3, at least one additional acoustic input and/or at least one non-acoustic input may be (requested and) received by the system. Similarly as mentioned above, the at least one additional acoustic input or the at least one non-acoustic input may be used as a basis for a (re-)evaluation whether the health state of the patient has (indeed) deteriorated. In such a case, it may be possible to determine (by the means for determining 5) whether the most recent at least one acoustic input is similar to any of the at least one stored acoustic input 4 and/or to an acoustic input which is stored in the at least one stored patient-specific profile. The means for determining 5 may follow a procedure as outlined above to re-evaluate the current health state of the patient based on the updated data basis.

In case a deterioration of the health state of the patient can be confirmed and/or if a determination of a deterioration is facilitated by means of the at least one additional acoustic input and/or by means of the at least one non-acoustic input, an indication may be provided to the patient and/or the medical service provider and/or to any other empowered person by means for indicating 7. The indication may be understood as an alert that the health state of the patient has deteriorated. The indication may be implemented as an acoustic alarm, a haptic alarm (e.g., a vibration) and/or a spoken sequence. The spoken sequence may e.g., relate to a computer voice which may articulate a warning that the health state of the patient has deteriorated. Additionally or alternatively, the indication may also automatically initiate a call to e.g., the medical care provider, an emergency center and/or to any other entity. The indication may also relate to a transmission, of the information that the health state of the patient has deteriorated, to e.g., an additional device which may e.g., be a user device of a doctor, a wearable and/or a smart home device and/or a medical device (e.g., a general surveillance device) and/or the indication may initiate a transmission to a hospital information system which may act as a database for the current health state and/or the time evolution of the health state of the patient. If the indication is provided to a smart home device, the smart home device may e.g., articulate a spoken sequence that the heath state of the patient has deteriorated and/or may indicate a deterioration by means of a flashing/blinking LED.

It may also be possible that the indication relates to suggesting the patient a certain action to recover the health state. Such a suggestion may e.g., comprise at least one of an administration of a pharmaceutical, a suggestion to pursue a certain sports activity and/or a suggestion to avoid certain nutrition habits.

In a preferred embodiment, the system as described above, is embodied as a smartphone. In such a case, the smartphone may receive a first acoustic input 2 and a second acoustic input 3. The first acoustic input 2 and/or the second acoustic input 3 may be received from e.g., a microphone. The microphone may be part of the smartphone or may be part of an external device, such as e.g., a smart home device, a wearable, a generic wireless connected device (e.g., a headset, a dedicated (medical) device for recording the first acoustic input 2 and/or the second acoustic input 3). Additionally or alternatively, the first acoustic input 2 and the second acoustic input 3 may be received from a database which is stored on the smartphone or a database which is located on a server in a remote computation center and/or a hospital.

The system, as described above, may e.g., be initiated and/or updated as a result of providing the patient with at least one question (which may relate to the health state of the patient and/or may be of a generic type, e.g., the weather). In response to the at least one question, the patient may answer the question and the respective response may be recorded. This may be understood as requesting 1 at least one acoustic input. It may be possible that the question is asked by a dedicated smartphone app, by a telemetric communication with a medical service provider and/or a smart home device and/or a wearable and/or a medical device or the question may appear on a display (e.g., of the smartphone and/or a smart home device). It may be possible that the patient is only provided with a single question. However, it may also be possible that the patient is provided with a plurality of questions which may be asked periodically and/or according to a predefined schedule or ad-hoc. In any case, the patient may be asked prior to starting any recordings to maintain the patient's privacy. The first and/or second acoustic input may then relate to the question.

The first acoustic input 2 and/or the second acoustic input 3 may be provided to the means for determining 5 to determine whether the health state of the patient has deteriorated. The means for determining 5 may either be part of the smartphone (e.g., in software) and/or may be a server-sided software solution (e.g., a cloud-based system). The means for determining 5 may also include an AI to determine whether the health state of the patient has deteriorated. The mans for determining 5 may compare the first acoustic input 2 with the second acoustic input 3. If it is assumed that the second acoustic input 3 has been recorded after the first acoustic input 2, any detectable difference of the first acoustic input 2 and the second acoustic input 3 may be interpreted as a change of the health state of the patient. Details on the comparison have been outlined above. Additionally or alternatively, it may also be possible that the smartphone compares the first acoustic input 2 and/or the second acoustic input 3 with at least one stored acoustic input 4 (e.g., as outlined above). The at least one stored acoustic input may also be stored on the smartphone and/or may be stored remotely (e.g., in a computation center) which may be accessible by the smartphone. Similarly as outlined above, the comparison may also be based on patient-specific profiles which may be stored on the smartphone and/or remotely.

However, it may also be possible that the means for determining 5 are part of a server-sided system such as e.g., a cloud-based system. In such an embodiment, the smartphone would transmit the first acoustic input 2 and/or the second acoustic input 3 to a respective server. The determination whether the health state of the patient has deteriorated may then be performed on the server. This may provide the advantage that less computational resources are occupied on the smartphone (which may affect the battery lifetime) and the determining may be faster when carried out on a performance-intensive computation system. The result of the determination of the server may then be transmitted to the smartphone. The smartphone may receive the result and may interpret the result as e.g., an indication in case the health state of the patient has deteriorated.

In case the means for determining 5 cannot find a similar health state as indicated by the first acoustic input 2 and the second acoustic input 3, among the at least one stored acoustic input 4, the means for determining 5, when embodied on the smartphone, may request at least one more acoustic input (as outlined above). Additionally or alternatively, the smartphone may also request at least one more non-acoustic parameter, e.g., a vital parameter. Said vital parameter may e.g., be obtained from a smartwatch which is (wirelessly) connected to the smartphone, an implant, a medical record as being stored in a hospital information system, etc. If the means for determining 5 are executed on a server, the server may request the at least one further acoustic input and/or non-acoustic input 6 from the smartphone (which may possess the above-mentioned opportunities to provide the respective input) and/or the server may request the respective information from a hospital information system.

In case a deterioration of the health state of the patient is determined by the means for determining 5, the smartphone may receive an indication (indicating the deterioration of the health state) from e.g., the remote server (if the system is implemented as a server-sided solution) or the respective algorithm (if the system is implemented as an app) and may provide the patient with a respective indication e.g., by a push notification message, optionally accompanied by an acoustic alert.

Alternatively or in addition, the smartphone may also forward the indication to a wearable device, e.g., a smartwatch. The smartwatch may indicate the deterioration of the health state of the patient by e.g., vibrating, an acoustic alert and/or flashing/blinking visual signal. Moreover, the smartphone may also be in connection with at least one smart home device such as e.g., a smart speaker (or a plurality of smart speakers). The smart speaker may articulate the deterioration of the health state of the patient by means of a spoken sequence. In any case, the indication may comprise a suggestion how the health state of the patient may be recovered. The suggestion may e.g., be based on an AI (based on similar health states and associated successful recovery strategies) or may be provided by a medical service provider.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

1. A system for determining a deterioration of a health state of a patient, the system comprising:
a) means for receiving a first acoustic input, associated with the health state of the patient at a first time, and a second acoustic input, associated with the health state of the patient at a second time;
b) means for determining whether the health state of the patient has deteriorated based at least in part on a comparison of the first acoustic input with the second acoustic input;
c) means for indicating the deterioration of the health state of the patient, if it is determined that the health state of the patient has deteriorated.

2. The system according to claim 1, further comprising means for requesting the first acoustic input and/or the second acoustic input.

3. The system according to claim 2, wherein the means for requesting is adapted to base a request on at least one of a predetermined schedule and/or an external input.

4. The system according to claim 1, further comprising means for receiving a value of at least one non-acoustic parameter associated with the health state of the patient.

5. The system according to claim 2, wherein the means for requesting the first acoustic input and/or the second acoustic input is adapted to request the first acoustic input and/or the second acoustic input if the value exceeds a threshold.

6. The system according to claim 1, wherein the means for receiving the first acoustic input and/or the second acoustic input comprises means for receiving a voice input and/or a respiratory sound input.

7. The system according to claim 1, wherein the system further comprises:
means for generating a patient-specific profile based at least in part on the first acoustic input and/or the second acoustic input; and
wherein the means for determining are adapted to determine whether the health state of the patient has deteriorated based at least in part on a comparison of the generated patient-specific profile with at least one stored patient-specific profile.

8. The system according to claim 4, wherein the means for generating the patient-specific profile further comprises means for including the value of the at least one non-acoustic parameter in the profile.

9. The system according to claim 1, further comprising means for providing the patient with a request for at least one additional acoustic input, if a deterioration of the health state of the patient is determined.

10. The system according to claim 1, wherein the first acoustic input and/or the second acoustic input is associated with a positivity value.

11. The system according to claim 1, further comprising:
means for analyzing the first acoustic input and/or the second acoustic input and/or the additional acoustic input,
wherein the means for analyzing comprises means for determining a pitch of a voice of the patient and/or a speech disruption and/or slow speaking and/or a vocal tremor and/or at least one repetition during speaking and/or a breathlessness, and/or means for converting the acoustic input into a storable text message.

12. A system for determining a deterioration of a health state of a patient, the system comprising:
a) means for transmitting a first acoustic input, associated with the health state of the patient at a first time, and a second acoustic input, associated with the health state of the patient at a second time, to an apparatus for determining whether the health state of the patient has deteriorated, based at least in part on a comparison of the first acoustic input with the second acoustic input;
b) means for receiving an indication of the deterioration of the health state of the patient in response to transmitting the first and second acoustic input, if, based at least in part on the comparison, it is determined, by the apparatus, that the health state of the patient has deteriorated.

13. A method for determining a deterioration of a health state of a patient, the method comprising:
a) receiving a first acoustic input, associated with the health state of the patient at a first time, and a second acoustic input, associated with the health state of the patient at a second time;
b) determining whether the health state of the patient has deteriorated based at least in part on a comparison of the first acoustic input with the second acoustic input;
c) indicating the deterioration of the health state of the patient, if it is determined that the health state of the patient has deteriorated.

14. A method for determining a deterioration of a health state of a patient, the method comprising:
a) transmitting a first acoustic input, associated with the health state of the patient at a first time, and a second acoustic input, associated with the health state of the patient at a second time, to an apparatus for determining whether the health state of the patient has deteriorated, based at least in part on a comparison of the first acoustic input with the second acoustic input;
b) receiving an indication of the deterioration of the health state of the patient in response to transmitting the first and second acoustic input, if, based at least in part on the comparison, it is determined, by the apparatus, that the health state of the patient has deteriorated.

15. A computer program comprising code which performs the steps according to any of the methods according to claim 13 when the code is executed.
